(19) United States
(12) Patent Application Publication
(10) Pub. No.: US 2006/0224089 A1
(43) Pub. Date: Oct. 5, 2006

(54) Method and apparatus for monitoring sleep behaviour

(75) Inventors: Kwong Luck Tan, Singapore (SG); Francis Tay Eng Hock, Singapore (SG); Hugo Van Bever, Singapore (SG)

Correspondence Address: BUCHANAN, INGERSOLL & ROONEY LLP, P.O. BOX 1404, ALEXANDRIA, VA, 22313-1404, US

(73) Assignee: Agency for Science, Technology and Research, Singapore (SG)

(21) Appl. No.: 11/156,317

(22) Filed: Jun. 16, 2005

Related U.S. Application Data
(60) Provisional application No. 60/666,410, filed on Mar. 29, 2005.

(51) Int. Cl. A61B 5/103

(52) U.S. Cl. 600/595

(57) ABSTRACT

An electric field mattress and a method for monitoring movement of a person are disclosed. The electric field mattress comprises a plurality of electrodes, a waveform generator electrically coupled to the electrodes for generating an electric field, and a detector coupled to at least one of the electrodes for detecting variations in the electric field. Variations in the electric field are caused by movement of a subject in the electric field.

### CROSS TO RELATED APPLICATION(S)

This application claims priority under 35 U.S.C. §119 to U.S. Provisional Application No. 60/666,410, filed Mar. 29, 2005, which is incorporated herein by reference in its entirety.

### FIELD OF THE INVENTION

The present invention relates generally to sleep behavior monitoring and more particularly to monitoring sleep behavior in subjects suffering from a condition that may affect their sleep patterns.

### BACKGROUND

Eczema, also known as atopic dermatitis, is a skin condition that affects people of all age groups but that is predominantly found in younger children and babies. The condition is exacerbated in regions having a tropical climate. For example, it has been estimated that 1 out of 10 individuals and 1 out of 5 children in Singapore suffer from eczema.

Although the severity of the condition may vary from person-to-person, the symptoms are similar and easily recognizable. Eczema patients experience inflamed skin rashes on parts of the body such as the scalp, face, groin, chest, arms and legs. Usually, affected areas are body parts having oil glands.

The rashes are intensely itchy and cause a patient to scratch incessantly in an attempt to alleviate their discomfort. Scratching activity continues during sleep periods and results in degradation of sleep quality, which may, in turn, lead to severe secondary effects. Monitoring of sleep behavior is thus highly beneficial for diagnosis and treatment of eczema patients.

Various devices and methods have been proposed for monitoring sleep quality of patients based on patient activity and/or physiological patient state. Devices for monitoring motion and activity during sleep can broadly be categorized into wearable devices, such as a watch or wristband, and devices that take the form of a bed.

An Actiwatch is a small data logging device for recording a digitally integrated measure of gross motor activity, which comprises a sensitive accelerometer, memory for recording data output by the accelerometer and an optional second input or sensor. Configuration of and data downloading from an Actiwatch device is performed by means of a data communications interface and a computer software program executed by a Personal Computer (PC). This provides a means for clinicians and researchers to measure sleep quality in individuals suffering from sleep disorders such as insomnia, PLM, sleep apnea, etc. An Actiwatch device is typically worn on the wrist or limb of an individual or patient, which disadvantageously may cause additional irritation to an eczema patient's skin. Another disadvantage is the high cost of an Actiwatch device and its associated software. Further information about Actiwatch may, for example, be obtained at the URL:<www.minimitter.com/Products/Brochures/900-0108-00_AW.pdf>

U.S. Pat. No. 6,485,441, entitled “SensorBed”, was published on 26 Nov. 2002 and relates to a device comprising a number of accelerometers and other sensors. This device is difficult to deploy and is not portable.

U.S. Pat. No. 5,796,340, entitled “Motion monitor useful for sleeping humans”, was published on 18 Aug. 1998. This document relates to a mattress having an isolated interior cavity with a pressure transducer therein for detecting respiration and/or cardiac activity of a patient.

U.S. Pat. No. 6,468,234, entitled “SleepSmart”, was published on 22 Aug. 1998. This document relates to a method and apparatus for measuring sleep quality that utilizes pressure and temperature sensors incorporated in a sheet, which can be laid on top of a conventional mattress. The apparatus comprises one or more layers of arrays of integrated pressure and/or temperature sensor pads for collecting data relating to physical properties such as an individual's position, temperature, sound, vibration and movement.

A need exists for improved methods and apparatuses for monitoring sleep behavior of a subject.

### SUMMARY

An aspect of the present invention provides an electric field mattress, which comprises a plurality of electrodes, a waveform generator electrically coupled to the electrodes for generating an electric field and a detector coupled to at least one of the electrodes for detecting variations in the electric field. The variations are caused by movement of a subject in the electric field. The electric field mattress may further comprise a multiplexer for selectively coupling individual ones of the electrodes to said detector. The plurality of electrodes may be disposed in a two or three dimensional array formation within the electric field mattress.

Another aspect of the present invention provides a method for monitoring movement of a person. The method comprises the steps of generating an electric field, detecting variations in the electric field, determining whether the variations represent movement of the person in the electric field and outputting an indication of a position of the person. The variations may be detected at a plurality of locations in the electric field that correspond to different areas of the person's body and may be indicative of movement of a limb of the person.

### BRIEF DESCRIPTION OF THE DRAWINGS

A small number of embodiments are described hereinafter, by way of example only, with reference to the accompanying drawings in which:

FIG. 1 is a graphical representation of sleep activity of a subject suffering from eczema;

FIG. 2a is a graphical representation of sleep activity of a subject free from eczema;

FIG. 2b is a graphical representation of sleep activity of a subject that suffers from eczema;

FIG. 3 is a block diagram of an electrode array for detecting motion and/or presence of a subject using an electric field;

FIG. 4 is a block diagram of another electrode array for detecting motion and/or presence of a subject using an electric field;

FIG. 5 is a block diagram of an apparatus for monitoring presence and/or movement of a subject using an electric field;

FIG. 6 is a flow diagram of a method for monitoring presence and/or movement of a subject using an electric field; and

FIGS. 7a, 7b and 7c show detection of presence of a human body on an electric field mattress according to an embodiment of the present invention.

### DETAILED DESCRIPTION

Embodiments of methods and apparatuses are described hereinafter for monitoring sleep behavior of a subject. Certain of the embodiments are described with specific reference to patients suffering from eczema. However, it is not intended that the present invention be limited in this manner as the principles of the present invention apply generally to monitoring of a subject's movements.

The present inventors conducted a series of experiments using commercially available Actiwatch devices to monitor sleep activity of various subjects who suffer and do not suffer from eczema.

In a first experiment, sleep activity of subjects suffering from eczema was monitored and analyzed using Actiwatch and compared to a video recording made of the subjects during the same monitoring period.

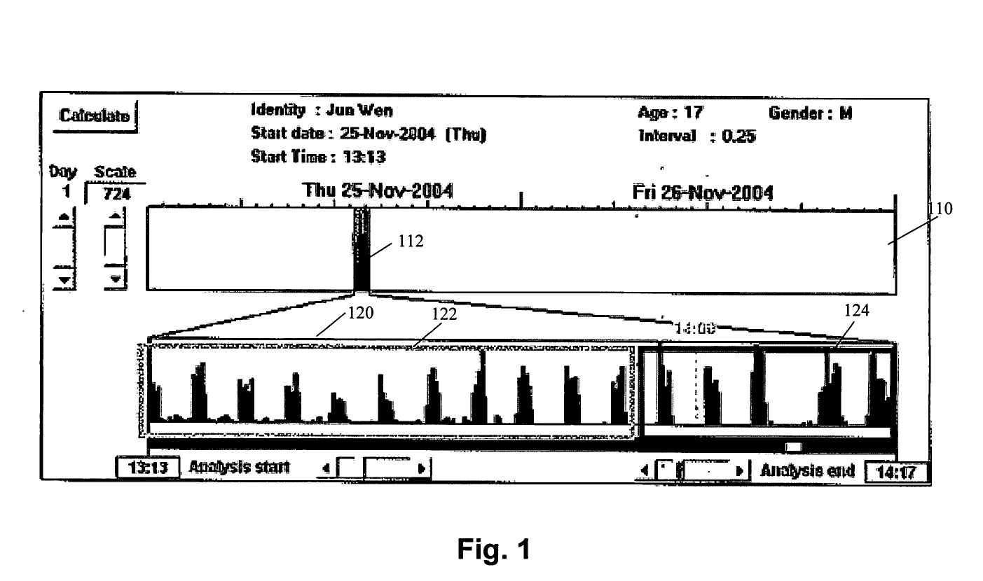

FIG. 1 shows graphs of sleep activity of a 17-year old subject suffering from eczema. Graph 120 is an exploded version of a portion 112 of graph 110. Window 122 of graph 120 shows a number of different scratching motions and indicates that scratching motions performed by different limbs are generally indistinguishable from each other. Window 124 of graph 120 shows movement of the whole body. Thus, scratching motions and movements of the whole body are generally indistinguishable.

In a second experiment, sleep activity of a subject free from eczema and sleep activity of a subject suffering from eczema were recorded over a 24-hour period using Actiwatch devices. The data was analyzed and compared against a video recording of the actual sleep activity of the respective subjects.

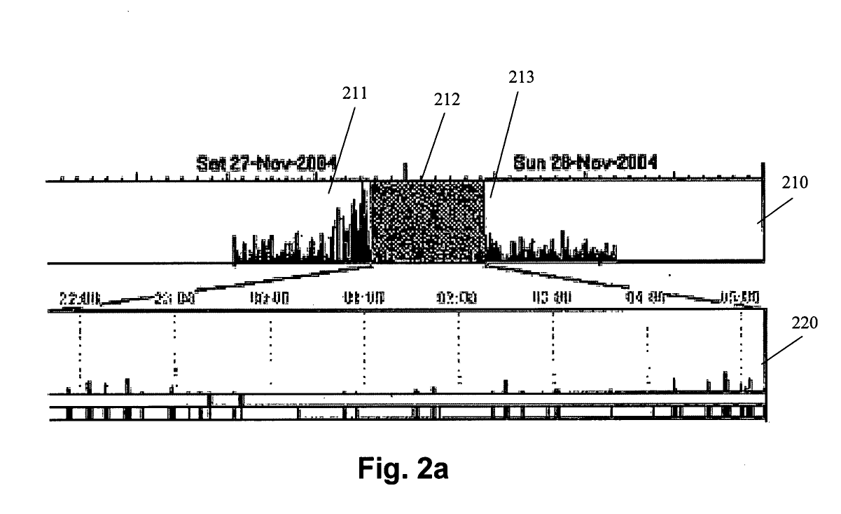

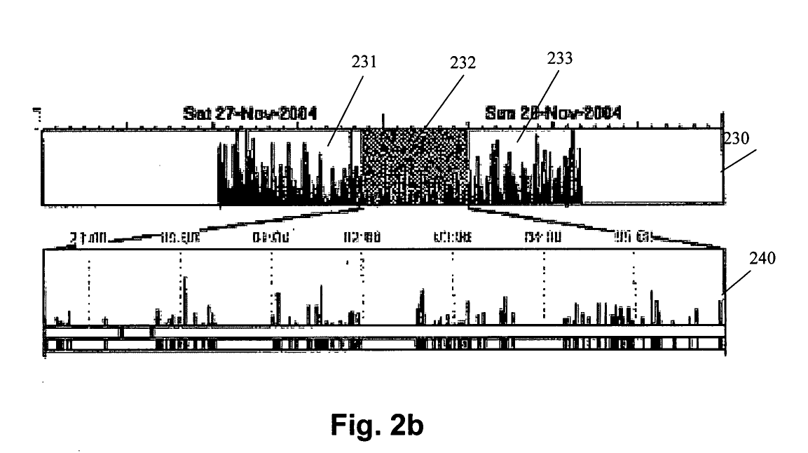

FIG. 2a shows graphs of sleep activity of a subject that is free from eczema. Graph 210 shows that the level of physical activity during the days (regions 211 and 213) is substantially greater than that during the intervening night (region 212). Graph 220 is an exploded version of region 212 in graph 210.

FIG. 2b shows graphs of sleep activity of a subject that suffers from eczema.

Graph 230 shows that the level of physical activity during the days (regions 231 and 233) is greater than that during the intervening night (region 232). However, the level of physical activity during the night is substantially greater for a subject that suffers from eczema than for a subject that is free from eczema (i.e., compare night regions 212 and 232 in graphs 210 and 230, respectively. Graph 240 is an exploded version of region 232 in graph 230.

The human body exhibits dielectric properties that can be detected using an electric field on account of comprising water containing ionic solutes. Advantageously, such detection can be performed using a low voltage electric field, which is not harmful to the human body.

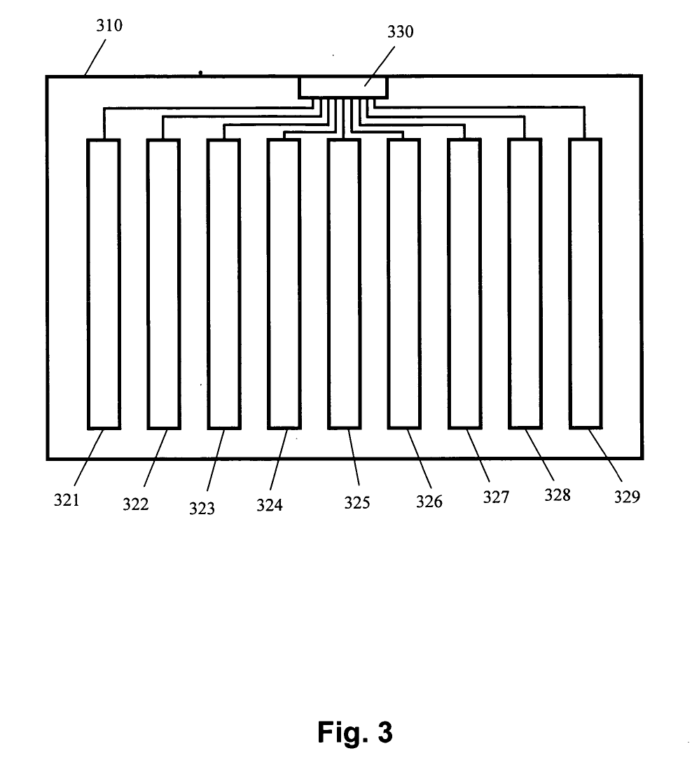

FIG. 3 shows an electrode array for detecting motion and/or presence of a subject using an electric field.

The electrode array 310 comprises a linearly disposed array of electrodes 321 . . . 329 electrically coupled via copper wires to an electrical connector 330. Each of the electrodes 321 . . . 329 are made from a laminated copper sheet but could alternatively be made from another electrically conductive material such as steel, aluminum and titanium. Also, the electrodes 321 . . . 329 may comprise a ferric mat or veil.

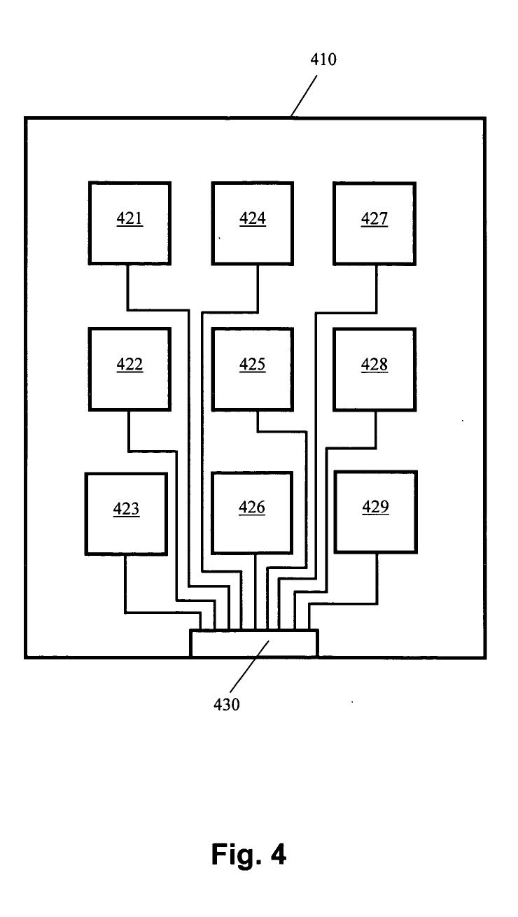

FIG. 4 shows another electrode array for detecting motion and/or presence of a subject using an electric field.

The electrode array 410 comprises a 3-by-3 array of electrodes 421 . . . 429 electrically coupled via copper wires to an electrical connector 430. Each of the electrodes 421 . . . 429 are made from a laminated copper sheet but could alternatively be made from other electrically conductive materials such as steel, aluminum and titanium. Also, the electrodes 321 . . . 329 may comprise a ferric mat or veil.

The electrode arrays 310 and 410 of FIGS. 3 and 4 each comprise 9 electrodes disposed in a two-dimensional grid formation. However, other numbers of electrodes may be practiced. In particular, a larger number of smaller electrodes results in improved detection of smaller or less conspicuous body movements of a subject. Moreover, multiple electrode arrays may be practiced in a stacked or three-dimensional formation to increase detection sensitivity.

The electrode arrays 310 and 410 of FIGS. 3 and 4 can be integrated with a conventional or custom mattress to provide an ‘electric field mattress’. Integration can be performed in numerous ways, including embedding of an electrode array into a conventional mattress or mounting of an electrode array on an upper or lower surface of a conventional mattress. Furthermore, an electrode array can be mounted on or embedded into a non-conductive base material, for example a soft fabric, which can be used in conjunction with a conventional mattress. Other non-conductive base materials may alternatively or additionally be practiced such as plastic, rubber and/or acrylics. Use of laminated copper sheet for the electrodes provides flexibility of the electrodes, which allows easier integration or embedding in a mattress and washing of the mattress.

In another embodiment, 28 electrodes in a 7×4 array, each of area approximately 10 cm², were sewn into a mattress protector for a single bed. The electrodes were energized by an electric field generator. A data logger with memory card storage was used to record raw 8-bit data representative of the magnitude of the electric field detected at the individual electrodes at regular intervals over a period of time. The data was transferred to a computer system for processing. Processing of the data identified variations in relation to the individual electrodes, which provided an indication of subject presence and movement over time. Detection of presence or absence of a limb was possible using the 7×4 array of electrodes.

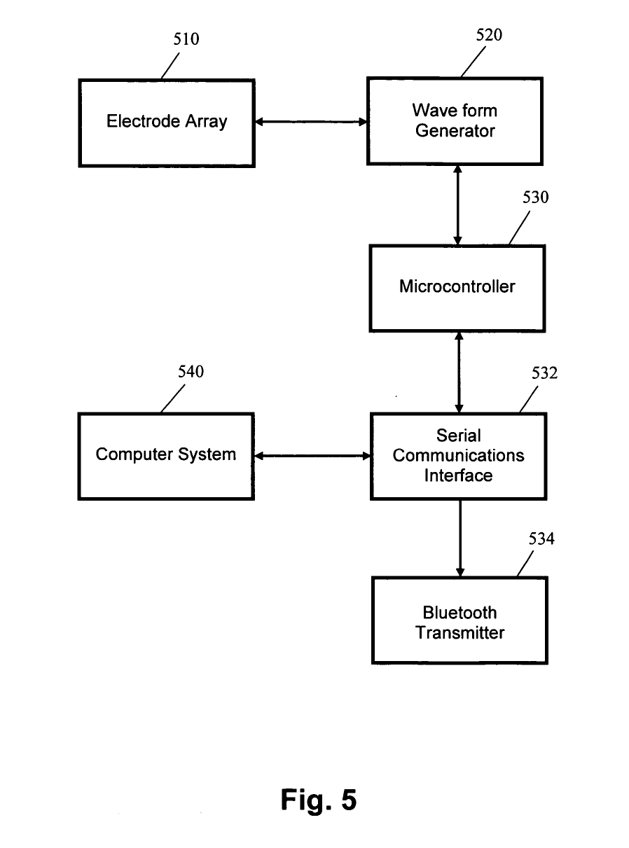

FIG. 5 shows a block diagram of an apparatus for monitoring presence and/or movement of a subject in an electric field. The apparatus comprises an array of electrodes 510, each energized by a waveform generator 520 coupled to the electrode array 510 to produce an electric field. Examples of electrode arrays used to practice the electrode array 510 include electrode arrays described hereinbefore in relation to FIGS. 3 and 4.

A microcontroller 530 is coupled to the waveform generator 520 to control the waveform generator 520 and to detect changes in the electric field generated by the waveform generator 520. The microcontroller 530 comprises memory, a communications interface and a processor for detecting and/or processing variations in the electric field. The microcontroller 530 is coupled to a serial communications interface 532, which is in turn coupled to a Bluetooth transmitter 534. The apparatus of FIG. 5 may be temporarily or permanently connected to a computer system 540 such as a personal computer (PC), laptop or notebook computer, personal digital assistant (PDA) via the serial communications interface 532 or wirelessly via the Bluetooth transmitter 534. Various serial or other communications interfaces and/or wireless communication means may be practiced, such as RS-232, RS-485, Universal Serial Bus (USB), FireWire, WiFi, etc. Data collected by the microcontroller 530 is transferred to the computer system 540 for storage and analysis. A software application executed by the computer system 540 enables analysis of the data from the electrode array 510 and presentation of results, including graphical representations of a subject's sleep activity and diagrammatic representations of a subject's position in relation to the electrodes.

In a particular embodiment of the present invention, the waveform generator 520 comprises a Motorola 33794 integrated circuit configured to operate at 120 kHz and the microcontroller 530 comprises a Motorola DSP56F8323 16-bit digital signal processor chip, which includes an on-board 16-bit analogue-to-digital converter. A generator internal to the Motorola 33794 integrated circuit produces a 5.0V peak-to-peak sine waveform having low harmonic content for driving the electrode array 510. A receiver-multiplexer internal to the Motorola 33794 integrated circuit simultaneously connects a currently selected electrode to a detector, also internal to the Motorola 33794 integrated circuit, which converts the sine wave to a DC signal. The DC signal is filtered, multiplied and offset to increase sensitivity and is then fed to an analogue-to-digital converter internal to the Motorola DSP56F8323 digital signal processor. The outputs of the remaining unselected electrodes in the electrode array 510 are internally grounded. A disturbance in the electric field is reflected as a capacitive voltage change at one or more of the electrodes. Such disturbances are detected and fed to the analogue-to-digital converter in the Motorola DSP56F8323 digital signal processor.

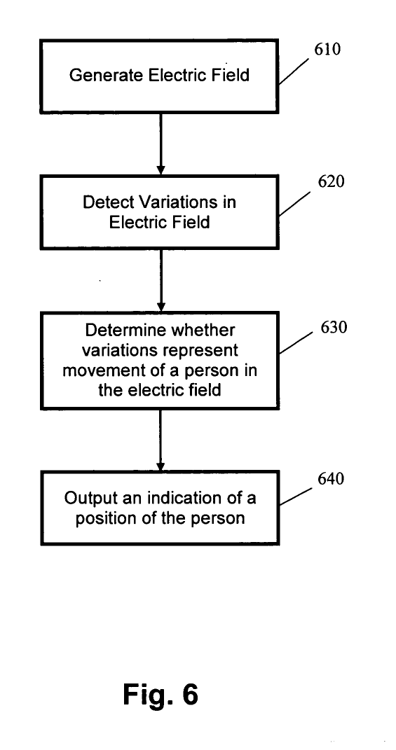

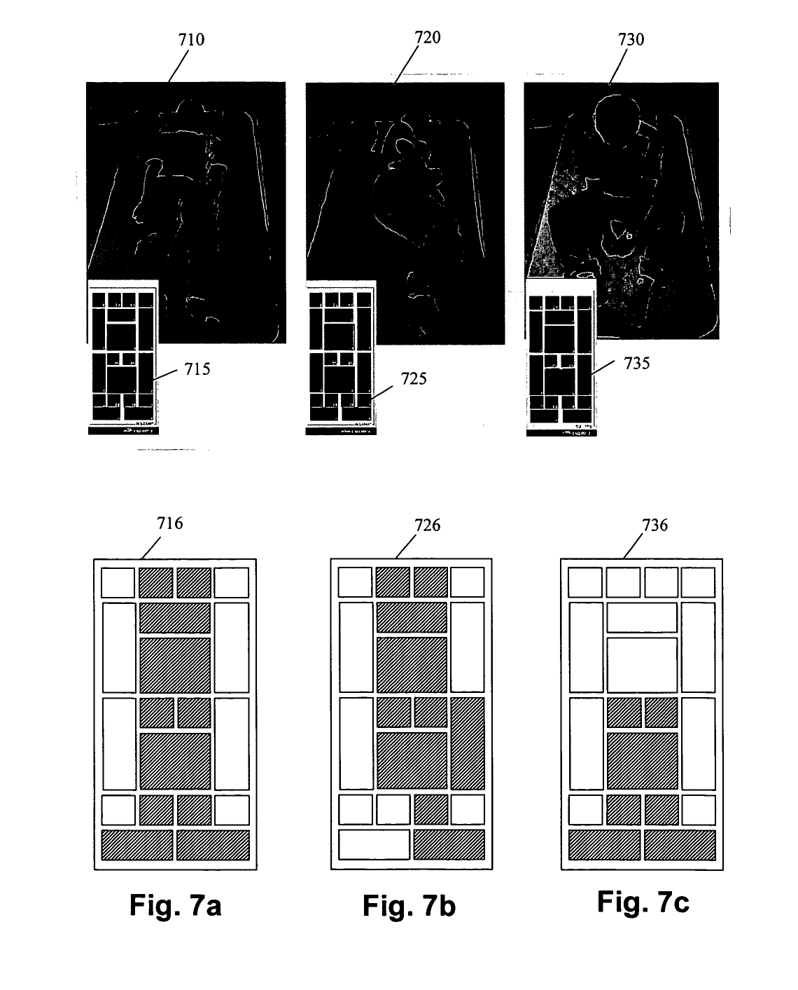

FIG. 6 shows a flow diagram of a method for monitoring position and/or movement of a subject using an electric field. An electric field is generated at step 610. Variations in the electric field are detected at step 620. At step 630, a determination is made whether the variations detected in step 620 are representative of position and/or movement of a subject in the electric field. An indication of subject movements and/or position is output at step 640. Examples of such output are shown in FIGS. 7a, 7b and 7c, hereinafter.

FIGS. 7a, 7b and 7c each show detection of presence of a human body on an electric field mattress according to an embodiment of the present invention.

FIG. 7a shows a graphic representation 710 of a person lying on their back on an electric field mattress and a corresponding graphical output 715 that indicates detection of the presence of the person's body by certain electrodes in an array within the mattress. A more detailed view 716 of the graphical output 715 indicates the detecting electrodes by way of cross-hatching.

FIG. 7b shows a picture 720 of a person lying on their left side on an electric field mattress and a corresponding graphical output 725 that indicates detection of the presence of the person's body by certain electrodes in an array within the mattress. A more detailed view 726 of the graphical output 725 indicates the detecting electrodes by way of cross-hatching.

FIG. 7c shows a picture 730 of a person sitting on an electric field mattress and a corresponding graphical output 735 that indicates detection of the presence of the person's body by certain electrodes in an array within the mattress. A more detailed view 736 of the graphical output 735 indicates the detecting electrodes by way of cross-hatching.

Advantageously, an electric field mattress according to embodiments of the present invention may economically be made to be foldable and portable.

The foregoing detailed description provides exemplary embodiments only, and is not intended to limit the scope, applicability or configurations of the invention. Rather, the description of the exemplary embodiments provides those skilled in the art with enabling descriptions for implementing an embodiment of the invention. Various changes may be made in the function and arrangement of elements without departing from the spirit and scope of the invention as set forth in the claims hereinafter.

Where specific features, elements and steps referred to herein have known equivalents in the art to which the invention relates, such known equivalents are deemed to be incorporated herein as if individually set forth. Furthermore, features, elements and steps referred to in respect of particular embodiments may optionally form part of any of the other embodiments unless stated to the contrary.

We claim:

1. An electric field mattress, comprising:
a plurality of electrodes;
a waveform generator electrically coupled to said electrodes for generating an electric field; and
a detector coupled to at least one of said electrodes for detecting variations in said electric field;
wherein said variations are caused by movement of a subject in said electric field.

2. The electric field mattress of claim 1, wherein said variations are caused by a change in dielectric in said electric field.

3. The electric field mattress of claim 2, wherein each of said plurality of electrodes is fabricated from laminated copper sheets.

4. The electric field mattress of claim 2, wherein said plurality of electrodes are disposed to form a two-dimensional array of electrodes.

5. The electric field mattress of claim 2, wherein said plurality of electrodes are disposed to form a three-dimensional array of electrodes.

6. The electric field mattress of claim 2, further comprising a multiplexer for selectively coupling individual ones of said electrodes to said detector.

7. The electric field mattress of claim 2, further comprising a computer system for processing the variations detected in said electric field to identify movements of said subject.

8. The electric field mattress of any one of claims 1 to 7, wherein said plurality of electrodes is disposed within said electric field mattress.

9. The electric field mattress of any one of claims 1 to 7, wherein said subject comprises a human being.

10. A method for monitoring movement of a person, said method comprising the steps of:
generating an electric field; and
detecting variations in said electric field;
determining whether said variations represent movement of said person in said electric field; and
outputting an indication of a position of said person.

11. The method of claim 10, wherein said variations are detected at a plurality of locations in said electric field that correspond to different areas of said person's body.

12. The method of claim 11, wherein said variations are indicative of movement of a limb of said person.

13. The method of claim 11, comprising the further step of outputting an indication of a movement of said person.

14. The method of claim 10, wherein said variations are caused by a change in dielectric in said electric field.
